(19) United States
(12) Patent Application Publication
(10) Pub. No.: US 2006/0067854 A1
(43) Pub. Date: Mar. 30, 2006

(54) Air revitalization methods and systems

(75) Inventors: Jean M. Andino, Gainesville, FL (US); Chang-Yu Wu, Gainesville, FL (US); David W. Mazyck, Gainesville, FL (US); Arthur A. Teixeira, Gainesville, FL (US)

Correspondence Address: EDWARDS & ANGELL, LLP, P.O. BOX 55874, BOSTON, MA, 02205, US

(73) Assignee: University of Florida, Gainesville, FL (US)

(21) Appl. No.: 11/097,990

(22) Filed: Mar. 31, 2005

Related U.S. Application Data
(60) Provisional application No. 60/558,393, filed on Mar. 31, 2004.

(51) Int. Cl. A61L 2/08

(52) U.S. Cl. 422/22; 422/23; 422/122; 422/292

(57) ABSTRACT

Systems are provided for revitalizing air and cleaning or purifying an enclosed area such as a building or vehicle interior that includes introducing a cleansing gaseous material into the area and treating the area with one or more photocatalysts. Systems of the invention can provide effective removal or degradation of both microorganisms and gaseous chemical pollutants.

The present application claims the benefit of provisional application 60/558,393 filed Mar. 31, 2004, which is incorporated by reference herein in its entirety.

### FIELD OF THE INVENTION

The invention relates generally to methods and systems for removal or degradation of gaseous and solid materials from a targeted area. More particularly, systems are provided for cleaning or purifying an enclosed area such as a building or vehicle interior that includes i) introducing ozone or other cleansing gaseous material into the area and ii) treating the area with one or more photocatalysts.

### BACKGROUND

Indoor pollution has been recognized as a serious health issue. Indeed, it has been reported that individuals presenting environmental associated symptoms more typically have been exposed to substances originating indoors, rather than outdoors. See, Indoor Air Pollution, U.S. Government Printing Office Publication No. 1994-523-217/81322 1994. The American Medical Association has reported that one-third of the U.S. national health bill is for causes directly attributable to indoor pollution, which may include various pathogens such as molds, bacteria, viruses, and the like. Among other things, the concentration of many pollutants including such pathogens can be significantly higher indoors relative to outdoor environments. See Indoor Air Pollution, supra.

The interior environments of various vehicles are particularly problematic. Various efforts have been reported to cleanse or otherwise remove pollutants such as exhaust materials, pathogens such as molds and fungus that may reside in ventilation systems, and the like. Prior approaches have included cumbersome and often ineffective cleaning systems. See, for instance, U.S. Pat. Nos. 5,221,292 and 5,954,577. See also Japanese Patent Applications 0303994; 02253662; 00157621; and 11299470.

Transmission of pathogens and other pollutants is of particular concern in environments that utilize recirculated air, i.e. air from a contained environment such as an aircraft interior that is processed (e.g. heated) and then redistributed to the contained environment. Significant concerns exist with the presence of pathogens in aircraft and other contained environments, including pathogens causing Severe Acute Respiratory Syndrome (SARS), tuberculosis and Sick Building Syndrome.

Prior approaches have employed filtration systems, including HEPA (High Efficiency Particulate Air) filters. In particular, HEPA filters have been employed for microbe removal from recirculating air systems. However, such filters merely trap pathogens and the harmful pathogens thereby can accumulate within the filter system. Pathogens accumulated on a filter bed can operate as a source of contamination for the environment and breeding base of infectious agents. HEPA filters also may be completely ineffective against pathogens that are of insufficient size to be trapped by the filter matrix.

It thus would be desirable to have new cleaning and purification systems. It would be particularly desirable to have new purification systems that would be useful for enclosed areas, such as a building or vehicle interiors.

### SUMMARY OF THE INVENTION

We now provide new methods and systems for cleansing enclosed areas such as the interior of a building or vehicle. Methods and systems of the invention can effectively remove or otherwise degrade a variety of undesired gaseous chemicals and pathogens from enclosed environments.

Preferred methods of the invention include i) introducing a cleansing gaseous material into an enclosed area and ii) treating the area with one or more photocatalysts such as a semiconductor material, e.g. titania. A particularly preferred treatment gaseous material is ozone, although other materials may be employed such as halogenated gases.

The tandem gaseous and photocatalyst treatments of the invention provide for an effective treatment mechanism for microorganisms and other pathogens that are suspended in the ambient air of an enclosed area or present on surfaces within the area such as surfaces of an air handling system, furniture, and the like. The systems and methods of the invention can destroy or otherwise render inert harmful pathogens that may be present in the targeted environment, thereby avoiding issues associated with filtration-based approaches such as accumulation of active pathogens on a filter surface and failure to remove small-sized microbes.

Indeed, methods and systems of the invention provide effective cleansing and purification without use of a HEPA filter, or other type of filtration system.

In typical methods and systems of the invention, ozone is introduced into an enclosed area under positive pressure, e.g., through a feed source that introduces the gas into the enclosed area or an apparatus that is present within the enclosed area and generates or otherwise releases the cleansing gas into the area. The cleansing gas treatment may be applied for extended time periods, e.g. a substantially continuous, prolonged exposure, although effective results can be achieved with only intermittent cleansing gas treatment.

Methods and systems of the invention further include treatment of resident air of an enclosed area with one or more photocatalysts, such as a semiconductor material. The photocatalyst treatment is coordinated with the cleansing gaseous treatment and preferably is commenced after an area has been exposed to the cleansing gas, although the photocatalyst treatment suitably may occur before or during exposure of an area to the cleansing gas. Apparatus of varying configurations may be employed to provide the photocatalyst treatment, including e.g. an apparatus that contains 1) a packed bed containing a purifying effective amount of one or more of the photocatalysts, 2) an activating radiation source such as an ultraviolet radiation source, and 3) means for flowing air resident in the enclosed area through or otherwise proximate to the photocatalyst bed. A fluidized bed of the photocatalyst(s) also can be effective.

As mentioned above, a variety of enclosed environments may be treated with a system of the invention, including interiors of buildings and vehicles.

Systems of the invention are particularly useful for cleaning of aircraft interiors. For instance, after completion of a flight, the empty aircraft interior can be exposed to the multiple treatments of the invention to reliably remove pathogens from resident air and interior surfaces.

Other aspects of the invention are disclosed infra.

### BRIEF DESCRIPTION OF THE DRAWINGS

FIG. 1 shows schematically a preferred process of the invention;

FIG. 2 shows an exemplary photocatalyst treatment system of the invention; and

FIGS. 3 and 4 depict preferred purification systems of the invention.

### DETAILED DESCRIPTION OF THE INVENTION

As discussed above, systems and methods of the invention will be particularly useful for revitalization of indoor air environments and surfaces. Enclosed areas that have been infiltrated with chemical or biological agents can be effectively treated in accordance with the invention.

Significantly, the tandem cleansing gas and photocatalyst treatments of the invention provide for effective revitalization of areas that are contaminated with pathogens and/or chemicals to yield benign products. For instance, system of the invention that employ ozone treatment can yield products such carbon dioxide, oxygen, and water.

More particularly, the treatment with a cleansing gas such as ozone can remove pathogens as well as noxious or otherwise undesired chemicals. Ozone treatment is especially effective for removal of chemicals that contain unsaturated moieties.

Following exposure to such a cleansing gas, preferred photocatalyst treatment in the presence of water vapor can produce hydroxyl radicals (OH) which will react with and degrade organic chemicals and a wide variety of pathogens to yield relatively inert materials such as water and carbon dioxide. The photocatalyst treatment also can degrade residual cleansing gases from a preceding exposure to more inert materials, e.g. the photocatalytic treatment can degrade residual ozone to molecular oxygen (O₂).

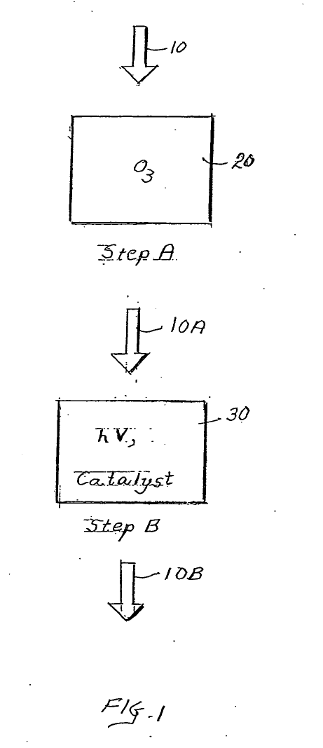

Referring now to the drawings, in FIG. 1 a preferred process of the invention is schematically shown. The area or material to be treated 10 is exposed to a gaseous cleansing material 20 such as ozone. In addition to ozone, other useful cleansing materials include e.g. a halogenated material, particularly a chlorinated material such as chlorine dioxide.

The area or material to be treated may be a variety of materials including e.g. air resident within an enclosed space such as a commercial or residential building, air resident within a vehicle such as a ground motor vehicle (car, truck, train, etc.), aircraft, storage or passenger compartments of a ship, submarine or other watercraft, military vehicles such tanks and the like, etc., and solid surfaces within such enclosed areas. Systems and methods of the invention will be particularly for treatment of air within enclosed spaces of medical facilities such as hospitals as well as governmental facilities where threatened or actual intentional introductions of pathogens may occur.

The targeted material or area may be treated with cleansing gaseous material in a variety of ways. For instance, a cleansing gaseous material may be introduced under positive pressure into an area to be treated. Thus, e.g., gaseous ozone can be introduced into an enclosed area such as a building room or vehicle interior by a feed line displacing and mixing with air resident within the enclosed area. As discussed above, ozone thereby can render effectively inert airborne pathogens such as viruses, bacteria, fungus, and the like as well as such pathogens that may reside on various surfaces within the area such as furniture, walls, floors, ceilings, etc.

The treatment gaseous agent generally can be introduced into a targeted area under a variety of conditions and achieve good cleansing/purifying results. Optimal conditions for any particular environment can be readily determined empirically, e.g. selected amounts and exposure times of one or more gaseous agents may be introduced into a targeted area and the decrease of pathogens before and after the introduction measured to thereby determine optimal treatment conditions. Preferred amounts of cleansing gas introduced into may area may vary with several factors such as size of an enclosed area being treated, air flow or exchange rate through the area to be treated, and the like. For many applications, it may be preferred to introduce an amount of the cleansing gaseous agent to the enclosed area in an amount of at least 0.1 to 1 volume percent of the enclosed area, although greater or lower amounts of the cleansing gaseous agent also may be suitably introduced. It also may be preferred to provide an active exposure time (i.e. time during which the cleaning gaseous agent is being introduced into the enclosed area) of at least 5 minutes after a 0.1 volume percent or greater amount of the gaseous material has been introduced into the area. As discussed above, longer exposure times also may be employed to provide a substantially continuous treatment with ozone or other cleansing gas. For many applications, a concentration of ozone gas within an enclosed area of at least about 10 to 15 ppm will be suitable with an exposure time of about 20 to 30 minutes.

The cleansing gas may be introduced to an area in a variety of ways. For example, in the case of ozone being used as the cleansing gas, an ozone-generating device may be placed within or otherwise proximate to an area being treated. Such devices are known and typically generate ozone through treatment of air with electrical discharge or relatively short wavelength radiation (e.g. ultraviolet radiation having a wavelength of less than about 254 nm).

As shown in FIG. 1, after treatment with a cleansing gaseous agent, the targeted material or area (reference 10A in FIG. 1) is further treated with one or more photocatalysts contained within apparatus 30 that can provide further cleansing effects and yield treated material 10B.

As discussed above, a variety of photocatalysts may be employed. Semiconductor materials are generally preferred, such as titania (TiO₂), ZnO, Fe₂O₃, and mixtures of such materials. Particularly preferred photocatalysts comprise titania, and even more preferred are titania/silica-based catalysts, e.g. where titania is present on a silicia substrate or within a silica matrix. Titania-silicia pellets can be produced through sol-gel techniques and have been found to be particularly effective. U.S. Patent Publication 2002/0187082 discloses additional photocatalysts that may be useful in systems of the invention.

More specifically, to form a preferred SiO₂—TiO₂ composite gel photocatalyst, suitably one or more acids, water, silica alkoxide (silica precursor), and a cosolvent are employed. Ratios of these materials may range e.g. from 0.11:1 up to 1.4:1 of the volume of silica precursor. During gelation, the silica can be doped with a commercially available photocatalyst, such as titanium dioxide. The titania percentage suitably can vary from 0.5% to 40% on a wt/wt basis. Mixed alkoxide synthesis can also be used to form a composite gel of SiO₂ and TiO₂ with a more homogeneous distribution of TiO₂. Various synthesis and aging steps can produce composites with pore sizes ranging from the microporous (<10 angstroms mean pore size) to macroporous (>50 nm mean pore size) as desired. Catalyst pellets can be suitably prepared through a mold process. See, for instance, the procedures of Example 1 below, which details the preparation of a preferred SiO₂—TiO₂ photocatalyst for use in the methods and systems of the invention.

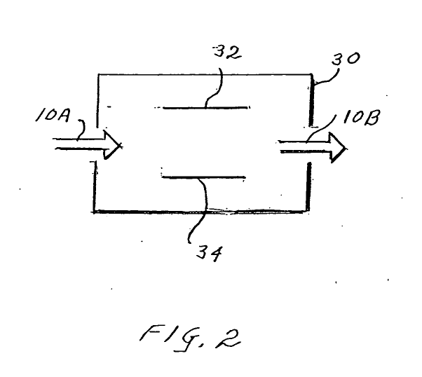

FIG. 2 illustrates schematically a suitable photocatalyst treatment apparatus in some greater detail. As depicted, material to be treated (reference 10A) passes into photocatalyst apparatus (reference 30), which may suitably contain a radiation source (reference 34) and photocatalyst bed (reference 32). A variety of radiation sources may be employed including e.g. an ultraviolet radiation source. As the admitted material (e.g. air resident in an enclosed area) passes through the photocatalyst apparatus, the bed of photocatalyst can be activated by the radiation source and react with the targeted material, particularly through generated hydroxyl radicals as discussed above, to degrade pollutants present in the material and then the purified material is passed from the apparatus.

In preferred systems, a packed bed of one or more photocatalysts is housed within the apparatus. Photocatalysts formed as discrete pellets or particles (i.e. separate and distinct particles or pellets) or as other packable configurations are preferred to provide such a catalytic bed. Additionally, porous pellets or particles can be particularly effective, e.g. catalytic pellets or particles that have a mean pore size from about 20 angstroms to about 500 angstroms, more typically a mean pore size of from about 30 angstroms to about 140 angstroms.

Rather than a packed bed, a fluidized photocatalyst system can be employed which can offer several advantages, including exposure of a greater volume of catalyst to activating radiation (e.g. a UV radiation source). The photocatalyst can be fluidized by a variety of methods, including mechanical agitation and use of a photocatalyst that contains a magnetic component and then exposure of the photocatalyst to a magnetic field to thereby provide agitation. Photocatalysts with a magnetic coating suitable for magnetic field agitation are disclosed in U.S. Patent Publication 2002/0187082.

Material to be treated (again, e.g., air resident within an enclosed area) can be drawn through apparatus by a variety of means including a fan or pump system. Suitable flow rates of material through the photocatalyst apparatus can vary rather widely. Optimal flow rates will vary with several factors, including the concentration and type of photocatalyst(s) within the photocatalyst apparatus, temperature and humidity of air passing through the apparatus, and the like. Preferred flow rates can be readily determined empirically.

A single or multiple photocatalyst apparatus may be employed to treat a targeted enclosed area. Multiple photocatalyst apparatus may be preferred to treat areas of larger volume such as large or multiple rooms of a building.

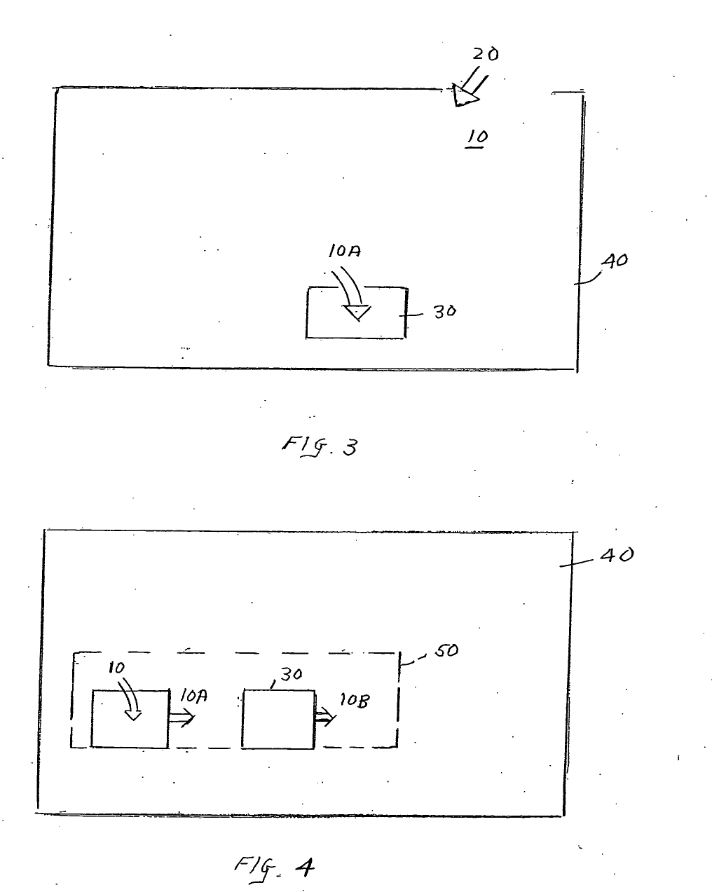

FIGS. 3 and 4 depict suitable approaches to treat an enclosed area with a system of the invention. Thus, FIG. 3 shows enclosed area (reference 40) which may be as discussed above one or more rooms of a building, interior of a vehicle, and the like. Cleansing gas (reference 20) such as ozone or a chlorinated gas is advanced into the enclosed area to treat resident air as well as exposed surfaces. After treatment of the targeted area for a desired period, introduction of a cleansing gas into the targeted area can be terminated. Prior to, at the same time as, or after treatment with the cleansing gas has been terminated, air within enclosed area can be treated with one or more photocatalysts with e.g. the depicted apparatus by flowing the treated air through the photocatalyst apparatus.

FIG. 4 depicts an alternatively configured system of the invention where gaseous and photocatalyst treatments are each housed within a single structure (reference 50). Air (reference 10B) resident within enclosed area 40 exits the photocatalyst apparatus 30 after successive ozone or other cleansing gas treatment and photocatalyst treatment.

All documents mentioned herein are incorporated herein by reference in their entirety.

The following non-limiting examples are illustrative of the invention.

### EXAMPLE 1

### Preparation of Preferred Photocatalyst for Use in Systems of the Invention

A preferred SiO₂—TiO₂ composite gel photocatalyst is formed using a sol-gel method. Acids of hydrofluoric acid and nitric acid, water, a silica alkoxide of tetraethyl orthosilicate (silica precursor), and cosolvent of ethanol are admixed and gelation induced. During gelation, the silica is doped with a commercially available photocatalyst, such as titanium dioxide. The titania percentage can vary from 0.5% to 40% on a wt/wt basis. When the solution becomes viscous, it is then pipeted into a mold in order to create a pellet of a certain size. After gelation, the composite is aged at room temperature for two days, then at 65° C. for two days. After aging, the pellets are removed from their mold, rinsed with water, and then placed in another container for additional heat treatments. The pellets are placed in an oven and the temperature is increased from room temperature to 103° C. and kept constant for 18 hours, resulting in vaporization of the liquid within the porous silica matrix to form a xerogel. The temperature is then increased to 180° C. and kept constant for 6 hours. Additional curing at higher temperatures can also be achieved (up to 600° C.) for strengthening of the gel. The resultant average pore size of the gel can range from a pore size of 30 angstroms to a pore size of between 100 to 200 angstroms, depending on the initial formula. The pellets can be used in a packed-column.

### EXAMPLE 2

### Operation of System of the Invention

A system of the invention corresponding to the configuration shown in FIG. 3 is provided by use of a commercially available corona discharge ozone generator that is positioned within the interior of a passenger aircraft that has been evacuated of passengers. The generator produces ozone within the aircraft for at least 20 minutes to a concentration of about 10 to 15 ppm. After such time, ozone generation is terminated, and a photocatalyst apparatus corresponding to the system 30 shown in FIG. 2 is operated to draw air resident within the airplane through the apparatus and proximate to a packed bed of titania-silica catalyst pellets produced as described in Example 1 above. The catalyst pellets are activated by exposure to an ultraviolet radiation source. Resident air is passed through the photocatalyst chamber for at least about 30 minutes.

The invention has been described in detail with reference to particular embodiments thereof. However, it will be appreciated that those skilled in the art, upon consideration of this disclosure, may make modifications and improvements within the spirit and scope of the invention.

What is claimed is:

1. A method for cleaning an enclosed area, comprising:
(a) introducing a cleansing gaseous material into the area; and
(b) treating the area with one or more photocatalysts.

2. The method of claim 1 wherein the gaseous material is ozone.

3. The method of claim 1 or 2 wherein the gaseous material is introduced to the area under a positive pressure.

4. The method of any one of claims 1 through 3 wherein air resident within the enclosed area flows through a structure containing the one or more photocatalysts.

5. The method of any one of claims 1 through 4 wherein air resident within the enclosed area is treated with a photocatalyst after treatment with the gaseous material.

6. The method of any one of claims 1 through 5 wherein the one or more photocatalysts are employed with a radiation source.

7. The method of any one of claims 1 through 6 wherein one or more of the photocatalysts comprise titania.

8. The method of any one of claims 1 through 6 wherein one or more of the photocatalysts comprise titania.

9. The method of any one of claims 1 through 6 wherein one or more of the photocatalysts comprise titania within a silica matrix.

10. The method of any one of claims 1 through 8 wherein the one or more photocatalysts generate hydroxyl radicals.

11. The method of any one of claims 1 through 10 wherein the area is the interior of a vehicle.

12. The method of claim 11 wherein the vehicle is an airplane, a ground vehicle, a military vehicle, ship or submarine.

13. The method of any one of claims 1 through 10 wherein the area is a room of a commercial or residential building.

14. A system for cleaning an enclosed area, comprising:
(a) an ozone source; and
(b) one or more photocatalysts.

15. The system of claim 14 wherein the ozone source and photocatalysts are positioned within or proximate to the enclosed area.

16. A system for cleaning an enclosed area, comprising:
(a) an ozone source; and
(b) one or more titania-silica photocatalysts that are coupled with an activating radiation source.

17. The system of claim 16 wherein the one or more photocatalysts are sol-gel titania-silica pellets.

18. The system of claim 16 or 17 wherein the one or more photocatalysts and radiation source are positioned within an apparatus.

19. The system of claim 18 wherein the apparatus comprises a packed or fluidized bed of the one or more photocatalysts and air resident in the enclosed area flows through or proximate to the catalyst bed.

20. The system of any one of claims 16 through 19 wherein the system is positioned within an aircraft.
